(19) United States
(12) Patent Application Publication
(10) Pub. No.: US 2020/0115332 A1
(43) Pub. Date: Apr. 16, 2020

(54) METHOD FOR THE PRODUCTION OF ALKANE SULFONIC ACIDS

(71) Applicant: GRILLO-WERKE AG, Duisburg (DE)

(72) Inventors: Timo Ott, Duisburg (DE); Christian Díaz-Urrutia, Duisburg (DE); Ingo Biertümpel, Duisburg (DE)

(21) Appl. No.: 16/483,523

(22) PCT Filed: Feb. 7, 2018
(86) PCT No.: PCT/EP2018/053081

§ 371 Date: Aug. 5, 2019

(30) Foreign Application Priority Data
Feb. 7, 2017 (EP) 17154935.5

(51) Int. Cl. C07C 303/06

(52) U.S. Cl. CPC C07C 303/06; C07C 309/04

(57) ABSTRACT

The present invention relates to methods for the production of alkane sulfonic acids, especially methane sulfonic acid, from alkane, especially methane, in which a carbocation, particularly a carbenium ion, is formed, as well as to the use of carbocations, particularly carbenium ions, for the production of alkane sulfonic acids, especially methane sulfonic acid.

The present invention relates to methods for the production of alkane sulfonic acids, especially methane sulfonic acid, from alkane, especially methane, in which a carbocation, particularly a carbenium ion, is formed, as well as to the use of carbocations, particularly carbenium ions, for the production of alkane sulfonic acids, especially methane sulfonic acid.

Alkanesulfonic acids are organic acids that can reach a similar acid strength as that of inorganic mineral acids, for example, sulfuric acid. However, in contrast to usual mineral acids such as sulfuric and nitric acids, the sulfonic acids are non-oxidizing and do not give off vapors that are harmful to health, as can be observed with hydrochloric and nitric acids. Further, many sulfonic acids, for example, methanesulfonic acid, are biologically degradable. The applications of sulfonic acids are many, for example, in cleaning agents, surfactants, as catalysts, and in organic synthesis, pharmaceutical chemistry, for example, as protective groups. The salts of sulfonic acids are employed, for example, as surfactants, for example, sodium dodecylsulfonate, or in the electroplating industry, especially as tin, zinc, silver, lead and indium, but also other metal, alkyl sulfonates. The very high solubility of alkyl sulfonates plays an important role, in particular. Further, no harmful gases are formed in electrolysis, and the use of toxic compounds, for example, cyanide, which is common in many cases, is dispensed with.

The structurally simplest representative of alkane sulfonic acids is methane sulfonic acid. Improvements of fracking techniques and biogas production have provided access to large quantities of inexpensive methane. Despite its abundance, methane is highly inert towards activation and functionalization to more complex molecules and, most importantly due to methane's high molecular stability, it is a higher contributor to climate change than CO₂.

During the past several decades there have been extraordinary efforts to overcome this challenge and various potential solutions have been developed. However, the industrial applicability of these processes is somehow restricted by economic constraints (for example, expensive catalysts, impractical scalability, among other problems). Moreover, a process capable of upgrading CH₄ must be robust and stable in order to handle large quantities of material at an industrial scale.

Liquefying methane to methanol is also impractical as a result of the different CO:H₂ ratio of syngas production of 1:3 (synthesis of methanol needs a CO:2H₂ mixture). Several homogenous methods have targeted the C—H activation of methane using transition metal complexes such as Hg, Pt, Ir, Rh, Ti, and Pd, among others. However, the functionalization to more complex molecules is still elusive. Three interesting functionalization approaches can be highlighted: 1) methanol; 2) methyl bisulfate (MBS); and 3) methanesulfonic acid (MSA). Direct synthesis of methanol is challenging due to the rapid overoxidation of methanol to more stable products and the need for harsh conditions. The Hg-mediated electrophilic C—H activation of CH₄ in H₂SO₄ medium at high temperatures affords SO₂ and MBS, whereas the latter could potentially be converted to methanol at high dilutions.

Early work on methanesulfonation led to the report of several radical initiators and additives for the activation of CH₄ to methane sulfonic acid (MSA) in fuming sulfuric acid. However, their reactions lack of high yield and good selectivity. Additionally, the reaction mechanism is still not yet entirely understood. Obvious problems arise in order to scale up these processes such as additional catalytic routes for SO₂ conversion, excessive large reactors and extremely high temperatures. Methane sulfonic acid (MSA), in contrast to methyl bisulfate (MBS), is considered a high value-added product and a green acid (for example, non-oxidant, low vapor pressure, bio-degradable, and so on) with uses in the pharma, electronic and cleaning industry.

Basickes et al. (Basickes, Hogan, Sen, J. Am. Chem. Soc. 1996, 11, 13111-13112) describe the radical-initiated functionalization of methane and ethane in fuming sulfuric acid. Temperatures of 90° C. and above are necessary and the yield of MSA is low.

Mukhopadhyay and Bell (Organic Process Research & Development 2003, 7, 161-163) report the direct sulfonation of methane at low pressure to methanesulfonic acid in the presence of potassium peroxydisphosphate as the initiator. In order to active the initiator, a temperature of 95° C. is chosen and the conversion rate of SO₃ is below 30%.

Lobree and Bell (Ind. Eng. Chem. Res. 2001, 40, 736-742) also studied the K₂S₂O₈-initiated sulfonation of methane to methanesulfonic acid. A radical mechanism is described and high temperature as well as low concentrations of initiator are required in order to achieve modest conversion rates of SO₃.

U.S. Pat. No. 2,493,038 describes the preparation of methane sulfonic acid from SO₃ and methane. US 2005/0070614 describes further methods for preparing methane sulfonic acid, and its application. The methods known in the prior art are in part complicated, cost-intensive, and lead to undesirable products because of the harsh conditions.

WO 2004/041399 A2 and U.S. Pat. No. 7,119,226 B2 both suggest a radical pathway and chain reaction for the production of methane sulfonic acid. In general, radical chain reactions usually result in undesirable side products, which even manifest themselves as disturbing inhibitors in the production of alkane sulfonic acids, which may lead to termination of the actual reaction for preparing the alkane sulfonic acid and further to impurities, formation of side products and poor yields based on sulfate trioxide and methane.

It is thus the object of the present invention to provide an industrial process for the valorization of alkanes to alkane sulfonic acids. A particular problem of the invention is to provide such a process for the valorization of methane to methane sulfonic acid (MSA). The process should avoid by-products and be equimolar with respect to the educts, sustainable within the meaning of the twelve principles of Green Chemistry as well as economically feasible.

In a first embodiment the problem is solved by a method for the production of alkane sulfonic acids, especially methane sulfonic acid, from an alkane, especially methane, and sulfur trioxide, comprising a step of reacting a carbocation of the alkane, especially a carbocation of methane, with sulfur trioxide such that the alkane sulfonic acid is formed.

Surprisingly it has been found that alkane sulfonic acids in high yields and with hardly any undesirable side products can be obtained when a carbocation of the alkane is employed. The inventive method particularly differs from methods of the prior art in that an ionic pathway is employed to produce alkane sulfonic acids. The inventive method thus circumvents the involvement of radical chain reactions, which usually lead to the formation of several different by-products. These by-products are not observed in the method according to the present invention.

Furthermore, the reaction can be carried out at temperatures, where radical chain reactions do not take place as observed in the prior art where high temperatures are required. The inventive process can for example be carried efficiently out at room temperature or at about 30° C.

The addition of substances promoting the decomposition of initiators to radicals or stabilizing said radicals is not required in the inventive process. Particularly, no such substances are added in one embodiment of the invention. Such substances including metal salts (e.g., Pt, Hg, Rh). They show detrimental side effects of triggering side reactions, which can be avoided by the present invention.

The alkane from which the carbocation is derived might be any alkane but is preferably methane, ethane, propane, butane, isopropane or isobutane. The employment of methane as alkane in the inventive process to produce methane sulfonic acid is especially preferred. In this way methane can be valorized and put to a good use.

A carbocation within the meaning of the present invention is any ion with a positively charged carbon ion bearing a +1 electric charge. Particularly, a carbocation may be derived from the alkane by the formal addition of H⁺ to a carbon atom of the alkane leading to a positively charged pentavalent carbon atom, which is denoted here as a carbonium ion. If the alkane is methane, the respective carbonium ion is methanium (CH₅⁺). The carbocation may be derived from the alkane by the formal elimination of H⁻ from a carbon atom of the alkane leading to a positively charged trivalent carbon atom, which is denoted here as a carbenium ion. If the alkane is methane, the respective carbenium ion is methenium (CH₃⁺).

In the following the inventive method, its preferred embodiments and the ionic pathway involved are described in more detail and particularly with respect to the functionalization of methane to methane sulfonic acid. As far as the description relates to methane and methane sulfonic acid, it is not meant to limit the scope of the invention to methane. The same considerations apply mutatis mutandis to the employment of other alkanes, which are also within the scope of the invention. Methane is merely chosen as an illustrative example, although it is also preferred as alkane. By replacing hydrogen atoms with alkyl substitutes, methane can be converted to other alkanes.

In a preferred embodiment of the inventive method, the carbocation is a carbenium ion, especially methenium (CH₃⁺). Preferably, the step of reacting the carbocation of the alkane with sulfur trioxide comprises the steps of:
i) Reacting a carbenium ion with sulfur trioxide to form an alkyl sulfite cation
ii) Reacting the alkyl sulfite cation with the alkane to from the alkane sulfonic acid and regenerate the carbenium ion.

For example, the inventive method might include the formation of CH₃⁺, afterwards reacting with SO₃, formation of H₃C—O—SO₂⁺, which afterwards reacts with the alkane being present in the reaction mixture, leading to the formation of alkane sulfonic acid, as depicted below:

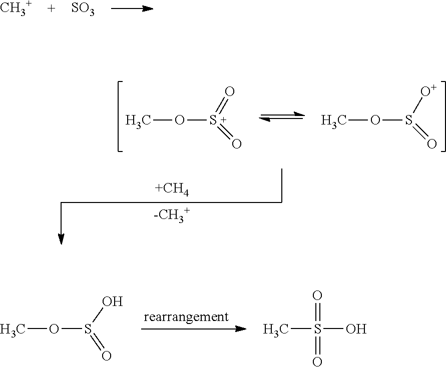

After producing some amount of carbenium ions, these ions can thus be employed in a catalytic cycle, wherein the direct reaction of sulfur trioxide with the alkane to the alkane sulfonic acid occurs via an ionic pathway.

The carbenium ion can be produced by any suitable method known from the prior art.

Particularly, the carbenium ion may be obtained by reacting the alkane with an activated pre-catalyst of the formula R³—O—O—R⁴, wherein the pre-catalyst comprises a hydrogen peroxide derivative and wherein the pre-catalyst is activated by reacting the pre-catalyst with a super acid.

A super acid is an acid with acidity greater than that of 100% pure sulfuric acid, which has Hammett acidity function (H₀ of −12). A super acid is thus a medium in which the chemical potential of the proton is higher than in pure sulfuric acid. Super acids include trifluoro methane sulfonic acid (CF₃SO₃H), also known as triflic acid, and trifluoro sulfuric acid (HSO₃F) or disulfuric acid (H₂S₂O₇). In the method according to the present invention, disulfuric acid is preferred as super acid. It might be obtained by reacting excess SO₃ with sulfuric acid.

R³—O—O—R⁴, can be any organic or inorganic peroxide suitable to be activated with a super acid and to react with an alkane to form a carbenium ion. Independently of being organic or inorganic, R³ and R⁴ might be the same or different from each other. Examples for suitable peroxides are peroxo carbonates, peroxo phosphates, peroxo sulfates and others.

Preferably, the pre-catalyst corresponds to the formula

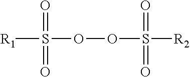

wherein R₁ and R₂ may be the same or different and are selected from the group of —H, —OH, —CH₃, —O—CH₃, —F, —Cl, —Br, —C₂H₅ or higher alkanes, —O—C₂H₅ or higher alkanes.

Especially, the pre-catalyst may be obtainable by reacting hydrogen peroxide with a sulfonic acid, especially methane sulfonic acid. A particular suitable pre-catalyst is asymmetric monomethyl sulfonyl peroxide (MMSP), which is obtainable by reacting methane sulfonic acid with hydrogen peroxide, optionally in H₂SO₄. The pre-catalyst may be prepared in situ by adding the precursor substances to the reaction mixture.

The pre-catalyst is activated by reacting it with a super acid. The activated pre-catalyst then reacts with the alkane to form a carbenium ion.

Preferably the pre-catalyst is employed in an amount of from 0.01 mol % to 30 mol %, based on the amount of sulfur trioxide. More preferably, the amount of the pre-catalyst is from 0.5 mol % to 5 mol %. Particularly the pre-catalyst may be employed in an amount of 0.9 mol %, based on the amount of sulfur trioxide.

As an example, the pathway described above are depicted below for the formation of methenium ions (CH₃⁺) from methane:

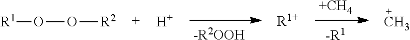

The invention, however, is not limited to this pathway. Preferably, the formation of the carbocation, particularly the formation of a carbenium ion, involves a reaction with a super acid. Preferably, the alkane is directly or indirectly reacted with the super acid. Preferably, the super acid is present in the reaction mixture.

In principle, suitable peroxo pre-catalysts could also react as radical initiators by homolytically breaking the —O—O— peroxo bond leading to two —O* radicals. Without the intention of being bound by theory, it is assumed that the pre-catalyst in the inventive ionic process is decomposed in a different way, i.e., not by breaking the O—O bond but rather by ionically spliting the R—O bond. After a proton is added to the O—O bond, leading to the formation of an R—O(H⁺)—O—R intermediate, the R—OH⁺ bond is broken, wherein R—OOH is formed and R⁺, which then reacts with an alkane to form a carbenium ion and a decomposition product of R.

Thus it is particularly preferred in the present invention that R¹ and R² are different, i.e., a non-symmetrical pre-catalyst is employed. In such a non-symmetrical pre-catalyst, the O—O bond is polarized which helps in protonating one of the oxygen atoms resulting in the heterolytic splitting of the peroxide. An example for such a non-symmetrical pre-catalyst is monomethyl sulfonyl peroxide

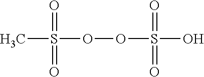

Sulfur trioxide can be used as pure SO₃ (100% SO₃) according to the present invention. This avoids the preparation of sulfur trioxide solutions. The reaction conditions are here without added solvents. Further, non-reacted sulfur trioxide can evaporate, avoiding the necessity of quenching it. Alternatively, sulfur trioxide can be used in a solution or as oleum with a trioxide content of 50% (w/w) or less, or 65% (w/w) or more. Surprisingly, it has been found that contrary to the prior art for the processes of the present invention also oleum with a sulfur trioxide content of 65% (w/w) or more, especially of 70% w/w or more can be used without negatively affecting the inventive process. Even pure sulfur trioxide (100% (w/w) sulfur trioxide) may be used. The sulfur trioxide content in solution of oleum is preferably within the range of from 15% (w/w) to 60% (w/w) and from 65% (w/w) to 99% (w/w), preferably of from 25% (w/w) to 60% (w/w) and from 70% (w/w) to 95% (w/w), especially preferred of from 35% (w/w) to 55% (w/w) and from 75% (w/w) to 90% (w/w). SO₃ contents below 15% (w/w) will also result in the formation of alkane sulfonic acids, but the reaction time will be so long that for economic reasons the reaction will become uninteresting. Surprisingly, it has been found that SO₃ contents of from 60% (w/w) to 65% (w/w) also have a very slow reaction time and are thus from an economical point of view not of interest.

The inventive method may be carried out in a reactor. Pure SO₃ or a solution containing sulfur trioxide or oleum is provided in the reactor. In the same reactor, an alkane, especially methane is provided. For alkanes with low boiling point, the use of a high pressure reactor is necessary. For pentane and higher alkanes, a common laboratorial reactor is sufficient. In the case of gaseous alkanes, for example methane, a pressure of 1 to 150 bar is set. For methane as alkane, the preferred pressure is within the range of from 10 to 150 bar, preferably of from 50 to 120 bar.

A super acid is provided in the reactor, for example by the addition of sulfuric acid into the reactor, where disulfuric acid is formed due to the presence of SO₃. If oleum is used as source for SO₃, no further super acid has to be added. Furthermore a pre-catalyst, as described above may be added. The pre-catalyst can also be added in form of a precursor, particularly in form of hydrogen peroxide and an alkane sulfonic acid, which will react with each order to form the pre-catalyst in situ in the reaction mixture.

The temperature of the reaction mixture is controlled to be within the range of from 0 to 100° C., preferably 0 to 50° C., leading to the formation of alkane sulfonic acid, especially methane sulfonic acid, dependent on the alkane provided as reactant. The resulting product, being the alkane sulfonic acid, might be purified, for example by distillation, crystallization, extraction or chromatography.

It is a particular advantage of the inventive process with respect to processes known from the prior art employing radical chain reactions, that a temperature may be chosen which is below the formation temperature of radicals. Preferably, the temperature is below 50° C., more preferably below 40° C., particularly below 35° C., especially below 30° C. The reaction may also be carried out at room temperature. With respect to the aforementioned upper bounds of preferred temperature range, a lower bound may particularly be room temperature.

Particularly preferred is the employment of the inventive method in the production of methane sulfonic acid (MSA) from methane and sulfur trioxide, which will be described in more detail in the following.

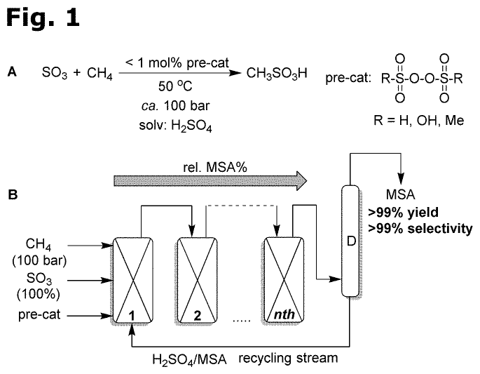

FIG. 1 shows the inventive method in its preferred embodiment for the activation and functionalization of CH₄ to MSA (see Part A of FIG. 1). Part B shows how continuous reactors (1, 2 and nth) in a pilot plant can produce up to 20 ton/year, the enriched mixture is then distilled in column D to obtain pure MSA. No by-products are observed and the H₂SO₄/MSA stream is recycled back to reactor 1.

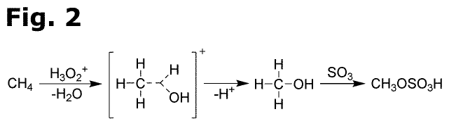

FIG. 2 shows the formation of a hydrogen peroxonium ion and the decomposition of methanol to MBS under superacid conditions.

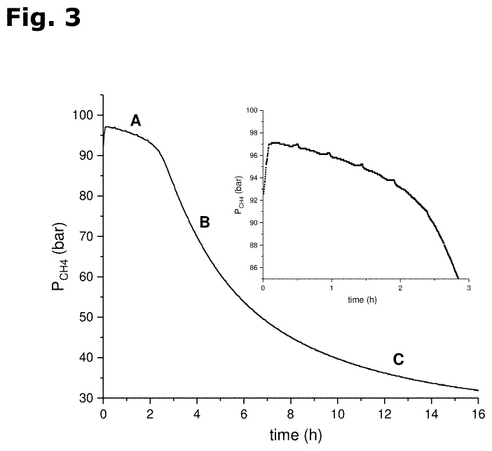

FIG. 3 (top) shows a reaction profile of the synthesis of MSA measured as pressure of CH₄ (bar) versus time (h); inset: close-up of region A. (bottom) shows a comparison of the reaction profile between the standard reaction and with the addition of traces of SO₂ as a deactivating agent.

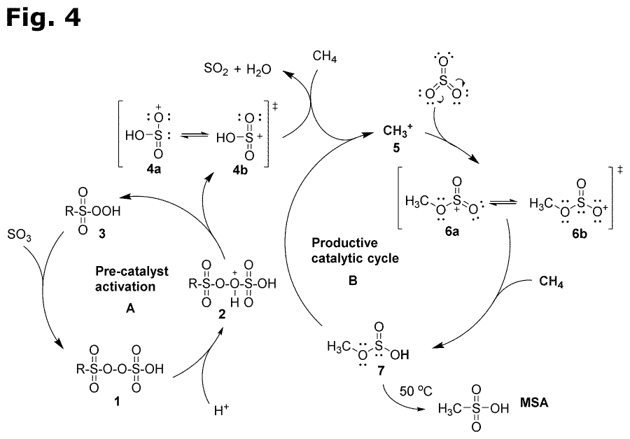

FIG. 4 shows the assumed cationic mechanism for the activation and functionalization of methane: A) pre-catalyst activation through the protonation of the peroxide 1 and; B) productive catalytic cycle where the methenium ion 5 is regenerated by the dehydrogenation of methane.

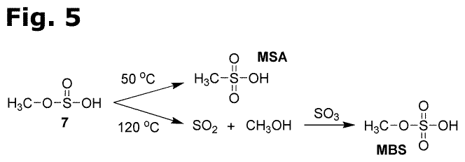

FIG. 5 shows how the disproportionation of methyl hydrogen sulfite at different temperatures affords MSA (50° C.) or MBS (120° C.).

A particular embodiment of the invention comprises the activation of methane at a pressure of circa 100 bar in a solution of fuming sulfuric acid (SO₃/H₂SO₄) of different concentrations (15 to 60%) with circa 1 mol % pre-catalyst comprising a hydrogen peroxide derivative (FIG. 1A). For production in a pilot plant, the reaction may be carried out in continuous reactors (FIG. 1B). Pure SO₃ and CH₄ are fed at the first reactor and then the reaction mixture is passed to the next one increasing the concentration of MSA until the nth reactor where the distillation takes place. The distillate consists of pure MSA with over 99% yield (based on the initial amount of SO₃) and 99% selectivity. The remaining solution comprising a mixture of H₂SO₄ and a small amount of MSA is fed back to the first reactor allowing for the desired oleum concentration at the beginning of the reaction. This configuration will allow scaling up the process to a major industrial production of around 10,000 metric tons of MSA/year.

400 mL batch reactors were used to carry out a detailed investigation of the reaction mechanism and the reaction optimization (Table 1). Diffusion of methane into the mixture is key in the conversions to MSA, propellers with gas diffusion must be used. Table 1 shows the yield of MSA under different conditions (for example temperature, pressure, et cetera) using 0.9 mol % pre-catalyst MMSP formed in-situ. The most common experiment is carried out with oleum 34% affording 60% yield of MSA in 16 h at 50° C. The yield of MSA is largely increased up to 99% using large reactors. Entry 3 depicts a reaction using an UV radiation (vidae infra) with no significant yield of MSA. The influence of SO₂ as deactivating agent is shown in Entry 4, where the total yield of MSA is 23%.

TABLE 1
Synthesis of MSA in a 400 mL batch reactor.

| | | | | Yield |
|---|---|---|---|---|
| Entry | SO₃ (%) | T (° C.) | P_{CH4} (bar) | MSA (%)^{a} |
| 1 | 34 | 50 | 97 | 60 |
| 2 | 24 | 50 | 97 | 83 |
| 3 | 24 | 25 | 96 | 0^{b} |
| 4 | 34 | 50 | 95 | 23^{c} |
| 5 | 36 | 50 | const.^{d} | 97 |
| 6 | 36 | 40 | 96 | 26^{e} |

^{a}percentage yield of MSA (analyzed by ion chromatography) based on initial amount of SO₃;
^{b}with UV light;
^{c}with addition of SO₂;
^{d}constant pressure of methane;
^{e}conversion based on the reacted amount of methane.

Particularly entry 3 of the above table shows that the reaction occurs via an ionic pathway. A radical chain reaction would be triggered by the UV light even at a temperature of 25° C. The UV light is capable of homolytically splitting peroxo bonds leading to the formation of radicals. Without the intention of being bound by theory it is assumed that said homolytic splitting in fact does take place and disables the ionic pathway, which presumably involves the heterolytic splitting of R₁—O—O—R₂ as discussed above.

Different compositions of pre-catalysts were studied. For example, solely H₂O₂ (60%) in H₂SO₄ (98%) does not trigger the formation of MSA in significant yields. Under superacid conditions H₂O₂ forms a hydrogen peroxonium ion H₃O₂⁺ that reacts with CH₄ that subsequently affords methanol (FIG. 2). A mixture of H₂O₂, MSA and H₂SO₄ exhibits the best performance towards the synthesis of MSA. The asymmetric monomethyl sulfonyl peroxide (MMSP) was identified in the pre-catalyst mixture. It is known in the prior art that symmetric dimethyl sulfonyl peroxide (DMSP) is capable of producing significant yields of MSA, however, selectivity and rates outperform with this pre-catalyst.

MMSP was identified via NMR, IR and MS. The following signals were obtained:
¹H NMR (neat H₂SO₄, capillary CDCl₃): δ 12.23 (ov s), 5.38 (ov s).
FT-IR (ATR cm⁻¹): 1693 (S—O), 1334 (S═O).
ESI-MS (m/z): 192.86 (MH−)

The reaction profile of the standard reaction (oleum 30%, 0.9 mol % of MMPS prepared in-situ, circa 100 bar of methane heating the reactor to 50° C.) shows a particular period of up to 2 h after the addition of the pre-catalyst where the pressure drop is almost linear (FIG. 3A). Afterwards, the decrease in pressure resembles a rapid decay (FIG. 3B) followed by a plateau after 10 h (FIG. 3C). The calculated yield of MSA based on the pressure drop, and supported by ion chromatography analysis, showed that equimolar amounts of product are formed relatively to the moles of H₂O₂ in the induction period (FIG. 3A). Moreover, the decomposition of the pre-catalyst at 50° C. and atmospheric pressure, measured by redox titration, rapidly occurs in the first 70 min with 40% decomposition of the peroxide. This indicates that the aforementioned period could be indeed the catalytic activation (FIG. 3A).

The reaction is highly sensitive to the temperature affording several product distributions and considerably affecting the rates. Low temperatures (below 50° C.) afford selectivities higher than 99% of MSA, however, at higher temperatures (above 100° C.) the reaction starts exhibiting more complex product mixtures with MBS and SO₂ as major components. Yields of MSA up to 85% can be obtained at 20° C. after seven days of reaction time. On the other hand, the concentration of SO₃ has important effects on the selectivity of MSA similar to those observed with change of temperature. At concentrations up to 60% of SO₃ the yields of MSA are quantitative, in contrast higher concentrations of SO₃ (>60%) promote the formation of MBS and SO₂, decreasing the yield and selectivity for MSA. Ethane, SO₂ and O₂, have also important effects as deactivating agents. For example, concentration of 1.29% and 0.44% (based on the total amount of SO₃) of SO₂ and C₂H₆, respectively, completely quenched the synthesis of MSA. The Hammett acidity values H₀ of different oleums increases with the amount of SO₃. For example, oleums of 35 mol % have H₀ values of −13.94, while the increase in acidity is consisted with the concentration of SO₃, however, at higher values over 50 mol % SO₃ the increase in acidity is very small (for example, for 75 mol % H₀=−14.96). This trend is in agreement with the observations in the synthesis of MSA where at higher SO₃ content the selectivity decreases with formation of high quantities of MBS and SO₂ at expenses of MSA.

Olah and co-workers (Olah, Prakash, Sommer, Molnar, Superacid Chemistry, Wiley-Interscience, 2.Auflage 2009) have extensively shown that under superacid conditions (for example, oleums of H₂SO₄) dissolved methane is protonated to a 2e-3c CH₅⁺ pentacoordinate cation. Similarly, H₂O₂ is protonated to generate a highly active hydrogen peroxonium ion H₃O₂⁺ which has been invoked in a large number of transformations. The rapid H/D exchange of CH₄ in D₂SO₄ at atmospheric pressure also demonstrates the facile C—H activation of CH₄ in a borderline superacid. The potential involvement of radicals in the formation of MSA under superacid conditions was tested. The irradiation of UV light with a broad wavelength Hg lamp is not sufficient to trigger the formation of MSA at 98 bar of CH₄ at room temperature with and without added pre-catalyst. On the other hand, control experiments showed that 0.9 mol % MMSP pre-catalyst prepared in-situ is capable to polymerize styrene at 25° C. under UV irradiation. In contrast, the polymerization of styrene is not observed in absence of UV light using the same pre-catalyst mixture.

Based on the described observations a cationic activation of methane followed by functionalization to MSA in superacid conditions is achieved, as depicted in FIG. 4.

Referring to FIG. 4, MMSP 1 is initially protonated to a peroxonium ion 2 which subsequently generates oxygen- and sulfur-centered cations (4a or 4b), and the hydroxyperoxide 3 that forms another molecule of MMSP 1 upon reaction with excess SO₃ (Scheme 1A). The species 4a or 4b activate CH₄ by electrophilic hydride abstraction to form a methenium ion 5. It is important to notice that the catalytic amount of MMSP 1 may be approximately 0.9 mol % based on the total amount of SO₃ and hence the amount of CH₃⁺ that enters the productive catalytic cycle in FIG. 4B. Nucleophilic attack of SO₃ on CH₃⁺ generates sulfur- and oxygen-centered methyl sulfite cations (6a and 6b) that resemble those formed in the pre-catalyst activation cycle. The methyl sulfite cation 6b can react with CH₄ to produce methyl hydrogen sulfite 7 that suffers a rapid rearrangement at 50° C. to MSA. Once the pre-catalyst MMSP 1 is completely consumed the productive catalytic cycle undergoes auto catalysis through the formation of CH₃⁺ 5. The assumed catalytic cycle takes into account the reaction profile observed in FIG. 3 with three different distinctive periods (vide supra). FIG. 5 shows the rearrangements of methyl hydrogen sulfite at different temperatures. High temperatures trigger the isomerization to SO₂ and methanol, the latter immediately reacts with free SO₃ to generate MBS.

The separation of MSA from the reaction mixture is challenging. Vacuum distillation achieved high purity of MSA, however, high temperatures afford decomposition products such as methane sulfonic acid anhydrous and methane sulfonic ester. Under these conditions, MBS is not observed as a decomposition product of MSA. Upon incorporation of the distillation into the continuous reactors; the present invention provides a highly efficient process for the mass production of MSA from only two reactants: SO₃ and CH₄.

In a further embodiment, the object of the invention is solved by the use of a carbocation for the production of an alkane sulfonic acid, especially methanesulfonic acid. Preferably, the carbocation is a carbenium ion, especially methenium (CH₃⁺). Particularly, the carbocation may be used for the production of methane sulfonic acid from methane and sulfur trioxide.

In a further embodiment, the object of the invention is solved by a method for the production of alkane sulfonic acids, especially methane sulfonic acid, comprising the following steps
i) Providing sulfur trioxide
ii) Providing an alkane, especially methane
iii) Providing a pre-catalyst, wherein the pre-catalyst comprises a hydrogen peroxide derivative
iv) Activating the pre-catalyst by reaction with a super acid, especially SO₃ in H₂SO₄
v) Reacting the pre-catalyst, the sulfur trioxide and the alkane in a reactor at a temperature of 50° C. or below
vi) Separating alkane sulfonic acid, especially methanesulfonic acid, from the reaction mixture.

Preferably, no substances promoting the formation of radicals or their stabilization are employed in the inventive process. Particularly, no metal salts are added to the reaction mixture.

Preferably, the pre-catalyst corresponds to the formula R₁—O—O—R₂, wherein R₁ and R₂ are different and optionally the peroxo bond in the pre-catalyst is polarized.

More preferably, the pre-catalyst corresponds to the formula

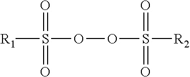

wherein R₁ and R₂ may be the same or different and are selected from the group of —H, —OH, —CH₃, —O—CH₃, —F, —Cl, —Br, —C₂H₅ or higher alkanes, —O—C₂H₅ or higher alkanes.

The pre-catalyst may be provided in step iii) by providing a mixture of hydrogen peroxide, an alkane sulfonic acid, especially methane, and sulfuric acid.

Preferably, the temperature in step v) is 40° C. or below, especially 30° C. or below, particularly 25° C. or room temperature.

In a preferred embodiment of the inventive method the pressure in step v) is within a range of from 10 to 150 bar, especially within a range of from 50 to 120 bar.

The sulfur trioxide may be employed in pure form or in a solution of sulfur trioxide in oleum, especially in a solution of 15 to 60% sulfur trioxide in oleum.

### EXAMPLES

### Example 1: Procedure for the Synthesis of MSA

In a 400 mL stainless steel high-pressure reactor, 245.02 g of fuming sulfuric acid (34.1%) were added using a HPLC pump maintaining the temperature of the lines at 50° C. The re-actor was heated to 50° C. with constant stirring speed of 1000 rpm. The pre-catalyst was prepared by dropwise addition of 464 μL of hydrogen peroxide (60%) over a cold mixture (0° C.) of 12 mL sulfuric acid (98%) and 1.38 mL MSA (99.5%). Once the reactor has reached a constant temperature of 50° C. the vessel is pressurized with 92.6 bar of methane (99.5%). The pre-catalyst was then injected into the rector using a HPLC pump raising the pressure inside the reactor to 97 bar. After 16 h the pressure dropped to 31.8 bar indicating that a large amount of methane was consumed. The reactor was then cooled down to room temperature, the excess pressure of methane was removed to a set of scrubbers and a sample consisting of a slightly colorless liquid was stored in a glass bottle weighing 279.57 g. The sample was subsequently analyzed using IC affording a 59.9% yield of MSA based on the total initial moles of SO₃.

### Example 2: Procedure for the Synthesis of MSA

In a 400 mL stainless steel high-pressure reactor, 288.07 g of fuming sulfuric acid 24% was added using a HPLC pump with constant heating of 50° C. The reactor was then heated to 50° C. and the stirrer was set to 1000 rpm. Once the temperature inside the reactor was constant at 50° C., 92.6 bar of methane were added. The pre-catalyst was prepared separately by dropwise addition of 464 μL of hydrogen peroxide (60%) over a cold mixture (0° C.) of 12 mL sulfuric acid (98%) and 1.38 mL MSA (99.5%). The pre-catalyst was added into the reactor using a HPLC pump increasing the total pressure to 97.4 bar. After 20 h of reaction time, the pressure dropped to 26.1 bar. The reactor was then cooled down to room temperature and the excess methane was removed to a set of scrubbers. The entire content (264.2 g) of the reactor was transferred to a glass bottle and stored properly. IC analysis showed that the yield of MSA in this reaction was 83.3% based on the total amount of SO₃ added.

### Example 3: Procedure for the Attempted Synthesis of MSA Using UV Radiation (Comparison Example)

In a 400 mL stainless steel high-pressure reactor equipped with two oblong sapphire windows, 249.31 g of fuming sulfuric acid 24% was added using a HPLC pump maintained at 50° C. The reactor was then heated to 25° C., the stirrer set to 1000 rpm and pressurized with 91.7 bar of methane. A medium-pressure mercury vapour UV lamp (UV-Consulting Peschl, Germany), broad emission over 190 nm equipped with a quartz immersion tube and a cooling jacket was positioned at 4 cm away from the reactor window, both covered with aluminum foil. The heat produce by the UV lamp was not enough to change the temperature inside the reactor. The pre-catalyst was prepared by dropwise addition of 464 μL of hydrogen peroxide (60%) over a cold mixture (0° C.) of 12 mL sulfuric acid (98%) and 1.38 mL MSA (99.5%). The pre-catalyst was then added into the reactor using a HPLC pump reaching a total pressure of 95.7 bar. After 2 h the temperature inside the reactor remained stable at 25° C. and the pressure was constant at 96 bar. At 4 h of reaction time the pressure still remained at 95.9 bar. The unchanged values in pressure indicate that the consumption of methane did not take place and MSA was not produced under these conditions.

### Example 4: Procedure for the Synthesis of MSA

In a 4 L stainless steel high pressure-reactor, 1.943 kg of fuming sulfuric acid 36% were added via cannula transfer. The reactor was maintained at 40° C. with a stirring speed of 350 rpm. Once the temperature is constant, the reactor is charged with 95.6 bar of methane. Separately, the pre-catalyst is prepared by dropwise addition of 3.4 mL of H₂O₂ (70%) over a cold mixture (0° C.) of 90 mL sulfuric acid (98%) and 10 mL MSA. The pre-catalyst is added into the reactor employing a HPLC pump raising the total pressure up to 98.5 bar. The pressure was constantly monitored during the experiment. At 16 h of reaction time the pressure was 71.1 bar. After 67 h the pressure inside the reactor has dropped to 31.1 bar. At this point the reactor was cooled down, the excess pressure of methane was removed to a set of scrubbers filled with sulfuric acid and a sample was taken. The sample was stored in a glass bottle weighting 2.244 kg. The calculated conversion of methane at 16 h (based on the initial amount of SO₃) was 26%. Ion chromatography showed that the yield of MSA after 67 h was 92% MSA.

1. A method for the production of alkane sulfonic acids from an alkane and sulfur trioxide, comprising a step of reacting a carbocation of the alkane with sulfur trioxide such that the alkane sulfonic acid is formed.

2. The method according to claim 1, wherein the carbocation is a carbenium ion.

3. The method according to claim 2, wherein the step of reacting the carbocation of the alkane with sulfur trioxide comprises the steps of
i) reacting the carbenium ion with the sulfur trioxide to form an alkyl sulfite cation, and
ii) reacting the alkyl sulfite cation with the alkane to form the alkane sulfonic acid and regenerate the carbenium ion.

4. The method according to claim 3, wherein the carbenium ion is obtained by reacting the alkane with an activated pre-catalyst, wherein the pre-catalyst comprises a hydrogen peroxide derivative and wherein the pre-catalyst is activated by reacting the pre-catalyst with a super acid.

5. The method according to claim 4, wherein the pre-catalyst corresponds to the formula 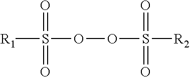
wherein R₁ and R₂ may be the same or different and are independently selected from the group of —H, —OH, —CH₃, —O—CH₃, —F, —Cl, —Br, —C₂H₅ or higher alkanes, and —O—C₂H₅ or higher alkanes.

6. The method according to claim 4, wherein the pre-catalyst corresponds to the formula R₁—O—O—R₂, wherein R₁ and R₂ are different and optionally the peroxo bond in the pre-catalyst is polarized.

7. The method according to claim 4, wherein the pre-catalyst is obtainable by reacting hydrogen peroxide with a sulfonic acid.

8. The method according to claim 1, wherein the sulfur trioxide is employed in pure form or in a solution of sulfur trioxide in oleum.

9. The method according to claim 1, wherein reacting a carbocation of the alkane—with sulfur trioxide is performed at a temperature within a range of from 0 to 100° C.

10. The method according to claim 9, wherein reacting a carbocation of the alkane—with sulfur trioxide is performed at a pressure within a range of from 10 to 150 bar.

11-12. (canceled)

13. A method for the production of alkane sulfonic acids comprising the following steps
i. Providing sulfur trioxide
ii. Providing an alkane
iii. Providing a pre-catalyst, wherein the pre-catalyst comprises a hydrogen peroxide derivative
iv. Activating the pre-catalyst by reaction with a super acid
v. Reacting the pre-catalyst, the sulfur trioxide and the alkane in a reactor at a temperature of 50° C. or below
vi. Separating alkane sulfonic acid from the reaction mixture

14. The method according to claim 13, wherein the pre-catalyst corresponds to the formula R₁—O—O—R₂, wherein R₁ and R₂ are different and optionally the peroxo bond in the pre-catalyst is polarized.

15. The method according to claim 13, wherein the pre-catalyst corresponds to the formula 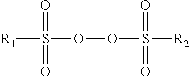
wherein R₁ and R₂ may be the same or different and are selected from the group of —H, —OH, —CH₃, —O—CH₃, —F, —Cl, —Br, —C₂H₅ or higher alkanes, and —O—C₂H₅ or higher alkanes.

16. The method according to claim 15, wherein the pre-catalyst is provided in step iii) by providing a mixture of hydrogen peroxide, an alkane sulfonic acid, and sulfuric acid.

17. The method according to claim 13, wherein the temperature in step v) is 40° C. or below.

18. The method according to claim 17, wherein the pressure in step v) is within a range of from 10 to 150 bar

19. The method according to claim 13, wherein the sulfur trioxide is employed in pure form or in a solution of sulfur trioxide in oleum.

20. The method of claim 1 wherein the alkane comprises methane, the alkane sulfonic acid comprises methanesulfonic acid, and the carbocation of the alkane is a carbocation of methane.

21. The method of claim 13 wherein the alkane comprises methane, the alkane sulfonic acid comprises methanesulfonic acid, the carbocation of the alkane is a carbocation of methane and the superacid comprises SO₃ in H₂SO₄.
